(19) United States
(12) Patent Application Publication
(10) Pub. No.: US 2026/0157873 A1
(43) Pub. Date: Jun. 11, 2026

(54) BILATERAL ARM STABILIZER SYSTEM

(71) Applicant: Freddie Drake McClendon, Manteca, CA (US)

(72) Inventor: Freddie Drake McClendon, Manteca, CA (US)

(21) Appl. No.: 19/350,772

(22) Filed: Oct. 6, 2025

Related U.S. Application Data
(60) Provisional application No. 63/704,012, filed on Oct. 6, 2024.

(51) Int. Cl. A61F 5/37

(52) U.S. Cl. CPC A61F 5/37

(57) ABSTRACT

A strap to assist with securing the arms of a medical patient during a lateral bed transfer of the patient is provided. The strap can have an anchoring patch on one end of the strap that adheres to an anchoring pad on the other end of the strap. The strap has a bridge portion formed by a notch cut into an edge of the strap. The bridge portion helps break the strap out of a hoop-like structure that is formed by joining the ends of the strap.

### CROSS-REFERENCES TO RELATED APPLICATIONS

This application claims priority to U.S. Provisional Application No. 63/704,012, filed Oct. 6, 2024, and entitled “BILATERAL ARM STABILIZER SYSTEM,” the disclosure of which application is hereby incorporated by reference herein in its entirety.

### BACKGROUND

Moving a medical patient laterally from one bed to another bed (e.g., to a transfer bed) can be difficult for numerous reasons. The patient is likely too heavy for a single caregiver to move the patient safely without injuring either the patient or the caregiver. Moreover, the patient may be unconscious or too weak to follow instructions that could assist with moving the patient safely. If a patient is unable to assist in the lateral move, the medical team that is moving the patient will likely need additional team members to assist in the move, or the move may take longer than it would if the patient were able to help with the move.

One problem with a lateral transfer of a patient is the problem of controlling the patient's arms, especially the arm that is closest to the transfer bed. If the patient is awake, oriented, and cooperative, one of the lift team members will usually ask the patient to fold the patient's arms onto the patient's chest and hug themselves. The transfer move then happens, and the patient's arms remain under control because the patient is hugging themselves, which keeps the patient's arms on the patient's chest and out of the way during the lateral move. If the patient is disoriented, unconscious, or under anesthesia, then another member of the lift team must hold and control the patient's arm until the transfer bed is brought close to the bed occupied by the patient. This requires an extra person in the lift team who must then stabilize both arms while the patient is moved onto the transfer bed. What is needed is a way to stabilize a patient's arms during a lateral transfer so that the move can be accomplished with less people, with less risk to the patient, and in a shorter amount of time. The device of the present disclosure is directed to solving these problems and does so in a quick, simple, and inexpensive way.

### SUMMARY

Certain embodiments of the disclosure comprise a strap that is easily attached and removed from a patient and configured to assist with securing the arms of the patient during a lateral bed transfer. In some arrangements, the strap can have an anchoring pad disposed on one end and an anchoring patch disposed on the other end. The anchoring pad and the anchoring patch can adhere to one another and keep the strap in a hoop-like structure that wraps around, and stabilizes, the arms of the patient. The strap can include a bridge portion that is formed by a notch that extends into the strap from a lateral edge of the strap. The bridge portion can help rupture the hoop-like structure to assist with removing the strap from the patient's arms.

In some aspects, the techniques and devices described herein relate to a method of laterally transferring a patient between adjacent beds.

### BRIEF DESCRIPTION OF THE DRAWINGS

Embodiments of the present disclosure will now be described hereinafter, by way of example only, with reference to the accompanying drawings in which:

FIG. 1 illustrates a bilateral arm stabilizing system applied to a patient prior to performing a lateral transfer of the patient, according to some aspects of the present disclosure.

FIG. 2A illustrates a bilateral arm stabilizing system in a strap configuration, according to some aspects of the present disclosure.

FIG. 2B shows the bilateral arm stabilizing system of FIG. 2A formed into a hoop-like structure, according to some aspects of the present disclosure.

FIG. 3A depicts a schematic representation of a side cross-sectional view of a bilateral arm stabilizing system mounted on two arms of a patient, according to some aspects of the present disclosure.

FIG. 3B depicts a schematic representation of a side cross-sectional view of a bilateral arm stabilizing system mounted on two arms of a patient, according to some aspects of the present disclosure.

FIG. 3C depicts a schematic representation of a side cross-sectional view of a bilateral arm stabilizing system mounted on two arms of a patient, according to some aspects of the present disclosure.

FIG. 4 depicts a bilateral arm stabilizing system stabilizing the arms of a patient, according to some aspects of the present disclosure.

FIG. 5 depicts a method of using a bilateral arm stabilizing system to assist with a lateral transfer of a patient, according to some aspects of the present disclosure.

### DETAILED DESCRIPTION

This disclosure relates generally to a quick-release device or system that can be used to stabilize the arms of a patient during a lateral transfer move. In some aspects, the system can be configured as a strap that adheres to itself to form a band or hoop-like structure that wraps circumferentially around both arms, or around both wrists, of the patient. The strap can include a weakened region or frangible portion that will fail under certain conditions (e.g., a force directed perpendicular to the strap) yet provide sufficient strength to remain intact under other conditions (e.g., a tensile force directed along the strap), as described herein. In some aspects, the strap can be configured to remain intact in a hoop-like structure when the strap is subjected to a small tensile load, such as that of a patient's arms falling laterally off the patient's chest. In some aspects, the strap can be configured to break when a hoop-like structure formed by the strap is subjected to a radially-outward-directed force, such a force that may occur upon a patient moving the patient's own arms voluntarily away from the patient's chest. In some aspects, the system can be comfortable for the patient because, for example, the patient can always elect to break the hoop-like structure and will not feel constrained, which could occur if the hoop-like structure did not easily break and the patient begins to regain consciousness but is not yet completely awake. In some aspects, the system can be helpful to the transfer team because, for example, the system is easy to apply to the patient and is sufficiently strong to serve its purpose of withstanding the small lateral forces of a patient's arms sliding off the patient's chest, which is likely to occur in the case of a lateral move of an unconscious patient.

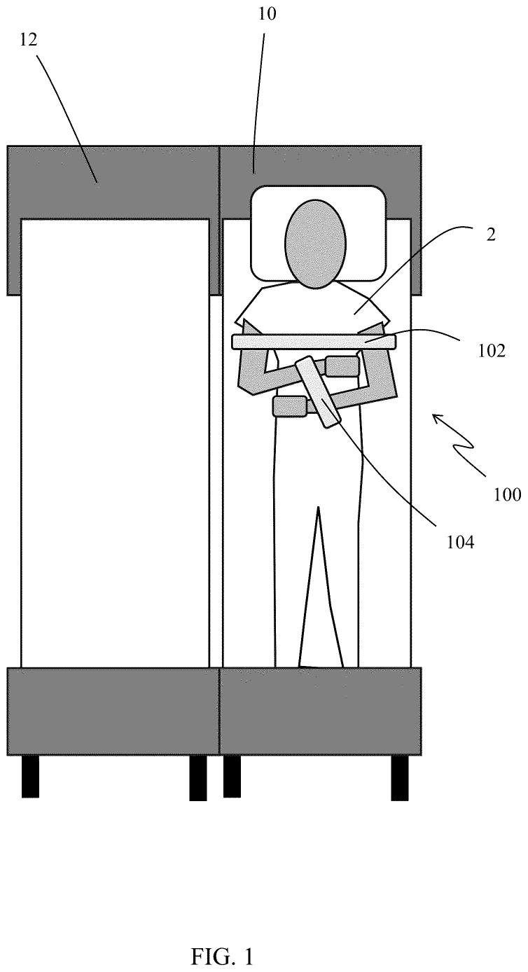

FIG. 1 depicts an illustrative, non-limiting example of a bilateral arm stabilizer system 100, according to some aspects of the present disclosure. In FIG. 1, a patient 2 is shown lying in a patient-occupied bed 10 and is prepared for a lateral transfer of the patient 2 from the patient-occupied bed 10 to a transfer bed 12. The patient's arms are positioned on the patient's chest. After this positioning of the patient, the bilateral arm stabilizer system 100 has been applied to the patient 2 and has been secured to keep the patient's arm on top of the patient's chest, as described herein. As shown in FIG. 1, the system 100 can be applied to a patient 2 at the upper arms (e.g., arm-applied system 102) or at the wrists (e.g., wrist-applied system 104). For the sake of simplicity, both the arm-applied system 102 and the wrist-applied system 104 will be referred to herein as the system 100. It can be appreciated, however, that the arm-applied system 102 and the wrist-applied system 104 may have differences, such as, for example, the arm-applied system 102 can be larger (e.g., longer, thicker) compared to the wrist-applied system 104.

In FIG. 1, the patient 2 is wearing both an arm-applied system 102 and a wrist-applied system 104. However, in some aspects, each of the arm-applied system 102 and the wrist-applied system 104 can be configured to stabilize the arms of the patient 2 without requiring the use of the other system 100. In other words, in some arrangements of the system 100, only one of either the arm-applied system 102 or the wrist-applied system 104 is needed to stabilize the arms of the patient 2.

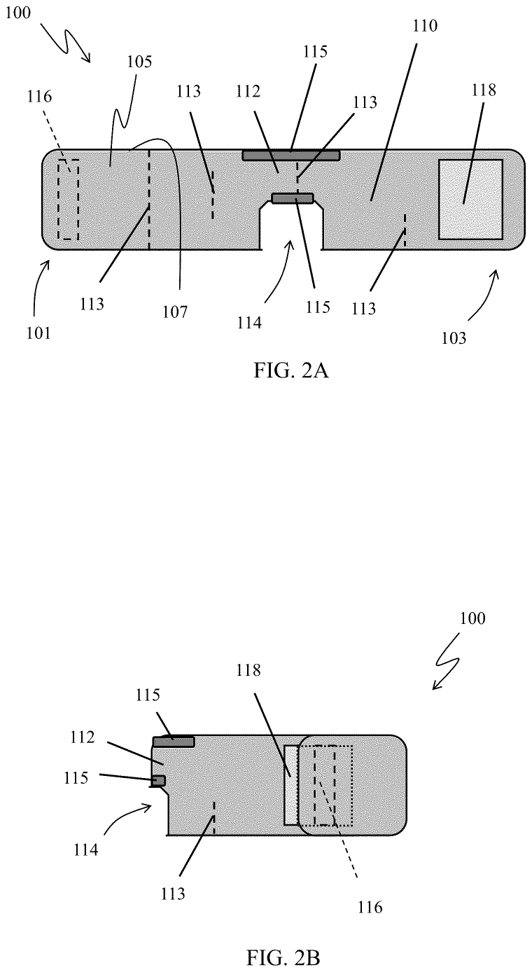

FIGS. 2A and 2B show a top view of a non-limiting, illustrative example of a bilateral arm stabilizer system 100, according to some aspects of the present disclosure. In FIG. 2A, the system 100 is shown in an initial configuration (e.g., before the system 100 has been applied to the patient 2). FIG. 2B shows the system 100 in a deployed configuration (e.g., when the system is being used on the patient 2 to stabilize the patient's arms). The system 100 is initially configured as a strip of material (FIG. 2A) that is passed between the arms and chest of a patient and then bent back on itself to form a hoop-like structure (FIG. 2B) that circumferentially sounds the patient's arms. FIG. 2B illustrates that the hoop-like structure of the patient-applied system 100 is maintained intact by an adhesive system (e.g., an anchoring patch 116 and an anchoring pad 118) that prevents the ends of the strap from moving relative to one another. In some aspects, the adhesive system can prevent or inhibit changes to the circumferential length of the hoop-like structure formed by securing the ends of the system 100 to one another with the adhesive system. Limiting the circumferential length of the hoop-like structure can, in turn, limit the ability of the patient arms to move within the hoop-like structure, thereby stabilizing the arms of the patient with the deployed system 100.

Turning to FIG. 2A, the system 100 can be configured as a strap 110 that has a head end 101, a foot end 103, a front surface 105 and a back surface 107, as indicated in FIG. 2A. The strap 110 can include a bridge portion 112 that spans a notch 114 formed by an absence of material along a lateral edge of the strap 110. In some arrangements, the notch 114 can be sized to promote rupture of the strap 110 at the bridge 112 (e.g., by increasing the magnitude of a tensile stress within the strap 110 at the reduced cross-sectional area of the bridge portion 112). In some arrangements, the bridge portion 112 can include a perforation 113 that promotes rupture of the strap 110 at the bridge portion 112, as shown in FIG. 2A. In some arrangements, the bridge portion 112 can include a re-enforced region 115 configured to bear, without rupturing, tensile stresses that would otherwise rupture the bridge portion 112. In some aspects, the re-enforced region 115 can prevent or inhibit the unintended rupture of the strap 110. In some aspects, the width of the bridge portion 112, the presence or absence of a perforation 113 on the bridge portion 112, and the presence or absence of a re-enforced region 115 can be tailored to provide sufficient tensile strength to the strap 110 when the system 100 is formed into a hoop-like structure to stabilize the arms of a patient. In some arrangements, the strap 110 can be configured to rupture at a location other than the bridge portion 112. For example, the strap 110 can include one or more perforations 113 disposed on the strap 110 at a location other than the bridge portion 112. FIG. 2A illustrates the strap 110 can include a perforation 113 disposed outside of the bridge portion 112 that extends across the entire width of the strap 110, or a perforation that extends only partially across the width of the strip 110.

In some aspects, the notch 114 can have a height dimension corresponding to the extent to which the notch 114 extends toward the midline of the strap 110 from a lateral edge of the strap 110. The notch 114 can have a length dimension corresponding to the extent to which the notch 114 extends along the lateral edge of the strap 110. In some arrangements, the notch 114 can be sized to approximate the width of a finger, allowing the notch 114 to serve as a location a person can position a finger to rupture the hoop-like structure of the deployed system 100, as described herein. For example, the notch 114 can have a width dimension of approximately two inches and a height dimension of approximately one inch. The hoop-like structure of the system 100 can be ruptured by inserting a finger into the notch 114 and pulling the bridge portion 112 radially outward relative to the hoop-like structure.

With continued reference to FIGS. 2A and 2B, the strap 110 can include an adhesive system for securing the head end 101 with the foot end 103 to form the hoop-like structure that surrounds and secures the patient's arms. In some arrangements, the adhesive system can include an anchoring patch 116 disposed at the head end 101 and configured to bind to an anchoring pad 118 disposed at the foot end 103. In some arrangements, the adhesive system can comprise an anchoring patch 116 or an anchoring pad 118 that adheres directly to the strap 110 without requiring both elements (patch 116 and pad 118) to secure the head end 101 to the foot end 103. Considering FIG. 2A along with FIG. 1, the strap 110 of FIG. 2A can be attached to a patient by positioning the strap 110 between the arms and chest of the patient with the front surface 105 facing the patient's arms, the head end 101 disposed longitudinally between the patient's head and arms, and the foot end 103 disposed longitudinally between the patient's feet and arms. The head end 101 can then be folded back towards the foot end 103 so that the strap 110 forms a hoop-like structure with the front surface 105 facing and circumferentially surrounding the patient's arms. In FIG. 2B, the hoop-like structure is formed by tucking the back surface 107 of the head end 101 radially inward of the front face 105 of the foot end 103 to bring the adhesive patch 116 into contact with the adhesive pad 118. Bringing the adhesive patch 116 and the adhesive pad 118 into contact with one another can cause the adhesive patch 116 to bind with the adhesive pad 118, locking the hoop-like structure to a fixed circumferential dimension and stabilizing the patient's arms, as described herein. To release the strap 110, an outwardly directed force can be applied to the hoop-like structure of the strap 110 (e.g., by a finger inserted under the bridge portion 112) causing the strap 110 to rupture (e.g., at a perforation 113), breaking the hoop-like structure and reverting the system 100 back to a strap-like structure.

FIGS. 2A and 2B show the bridge portion 112 can be disposed approximately mid-length of the strip 110, in some arrangements of the system 100. However, the bridge portion 112 can be disposed anywhere along the length of the strip 110 between the adhesive patch 116 and the adhesive pad 118 and need not be disposed mid-length as shown in FIG. 2A. FIGS. 2A and 2B also show the notch 114 is disposed on only one side of the strip 110 and extends to approximately halfway across the width of the strip 110. However, the bridge portion 112 can be formed by notches 114 disposed on opposing lateral sides of the strip 110. The opposing notches can be, but need not be, longitudinally aligned with one another. Further, the notch 114 can extend from a lateral side to a distance that is less than, or that is greater than, the mid-width dimension of the strip 110. The bridge portion 112 need not include a notch 114 disposed on a lateral side of the strip but can be configured instead include a series of slits, or windows, or perforations of missing material that are distributed across the width of the strip 110. The strap 110 can be made of paper or other suitable material that is lightweight and inexpensive. In some aspects, the strap 110 can be intended for one-time use.

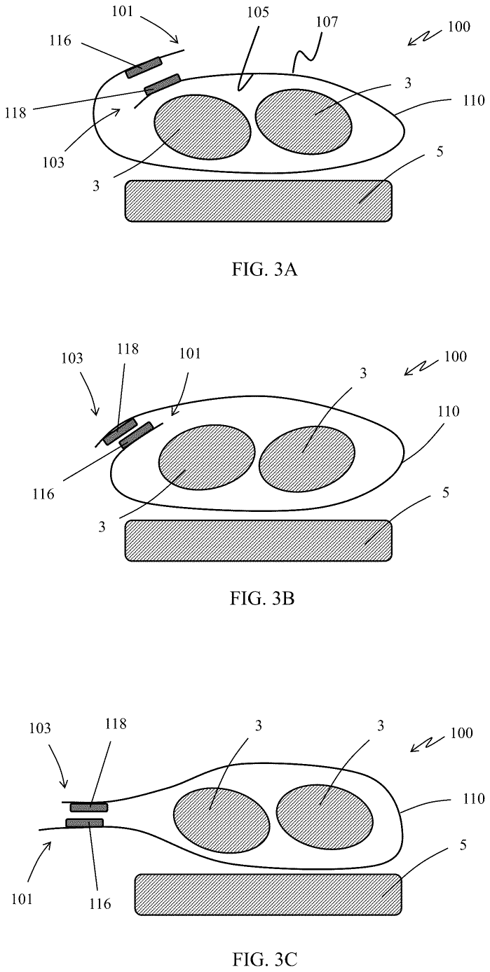

FIGS. 3A-3C show side views of a bilateral arm stabilizer system 100 that has been applied to secure a patient's arms 3 on top of the patient's chest 5, according to some aspects of the present disclosure. The labeling of the head end 101 and the foot end 103 in FIGS. 3A-3C reflects that for these figures the orientation of patient is with the patient's head to the left of the figure and the patient's feet to the right of the figure. FIGS. 3A-3C illustrate that the system 100 can use alternative arrangements of the adhesive patch 116 and the adhesive pad 118 to achieve securement of the head end 101 with the foot end 103 to maintain the hoop-like structure that secures the patient's arms 3 (e.g., during a lateral transfer of the patient). FIG. 3A illustrates the hoop-like structure can be formed by bringing an adhesive patch 116 disposed on the front face 105 into contact with an adhesive pad 118 disposed on the back face 107, with adhesive patch 116 disposed radially outward of the adhesive pad 118. FIG. 3B illustrates the hoop-like structure can be formed by bringing an adhesive pad 118 disposed on the front face 105 into contact with an adhesive patch 116 disposed on the back face 107, with the adhesive pad 118 disposed radially outward of the adhesive patch 116. FIG. 3C illustrates the hoop-like structure can be formed by bringing an adhesive patch 116 disposed on the front face 105 into contact with an adhesive pad 118 disposed on the front face 105. In some arrangements, the adhesive patch 116 can include one of a hook portion or a loop portion of a hook-and-loop fastening material with the adhesive pad 118 comprising the other of the hook portion or the loop portion such that the adhesive patch 116 secures to the adhesive pad 118 when the two are brought into contact. In some arrangements, the adhesive patch 116, or the adhesive pad 118, or both the adhesive patch 116 and the adhesive pad 118, can comprise an adhesive material (e.g., glue) that secures the adhesive pad 116 to the adhesive pad 118 when the two are brought into contact with one another.

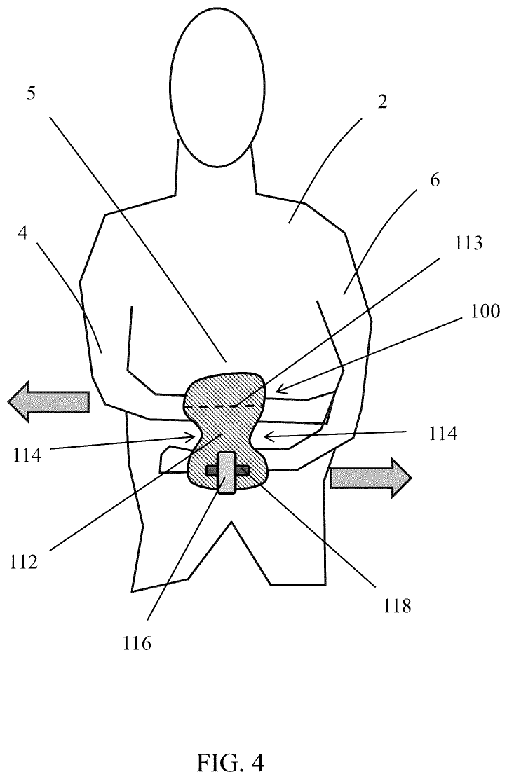

FIG. 4 shows a non-limiting, illustrative example of a system 100 in use on a patient 2, according to some aspects of the present disclosure. The system 100 is shown in its deployed configuration in which the system 100 forms a band (also referred to herein as a hoop-like structure) that wraps around both the wrist of the right arm 4 and the wrist of the left arm 6 of the patient 2. As described herein, the bridge portion 112 can be configured to withstand small lateral forces (indicated in FIG. 4 by the two, oppositely-directed, filled arrows) that arise when the arms 4, 6 of the patient 2 slide laterally off of the chest 5 of the patient 2. As can be appreciated from FIG. 4, these small lateral forces will tend to place the bridge portion 112 in slight tension. These small lateral forces will also tend to twist the bridge portion 112 and distribute the small tensile force obliquely over the bridge portion 112. The bridge portion 112 can be configured to withstand slight tension distributed obliquely across the bridge portion 112, as described herein, allowing the system 100 to maintain the hoop-like structure and stabilize the arms 4, 6 on the chest 5 of the patient 2. By contrast, should the patient 2 move the right arm 4, or the left arm 6, or both arms 4, 6 in an upward direction (which would be expected in a patient-voluntary motion), then the bridge portion 112 will be subjected to a larger force that is not obliquely distributed across the bridge portion 112, thereby causing the bridge portion 112 to fail and the hoop-like structure of the system 100 to pop open and release the arms 4, 6 of the patient 2. Similarly, once the lateral transfer move is completed, the hoop-like structure of the system 100 can be easily removed from the patient 2 by simply pulling radially outward on the hoop-like structure, breaking the bridge portion 112, and turning the system 100 back into a strap 110 configuration that can be easily pulled off of the patient 2.

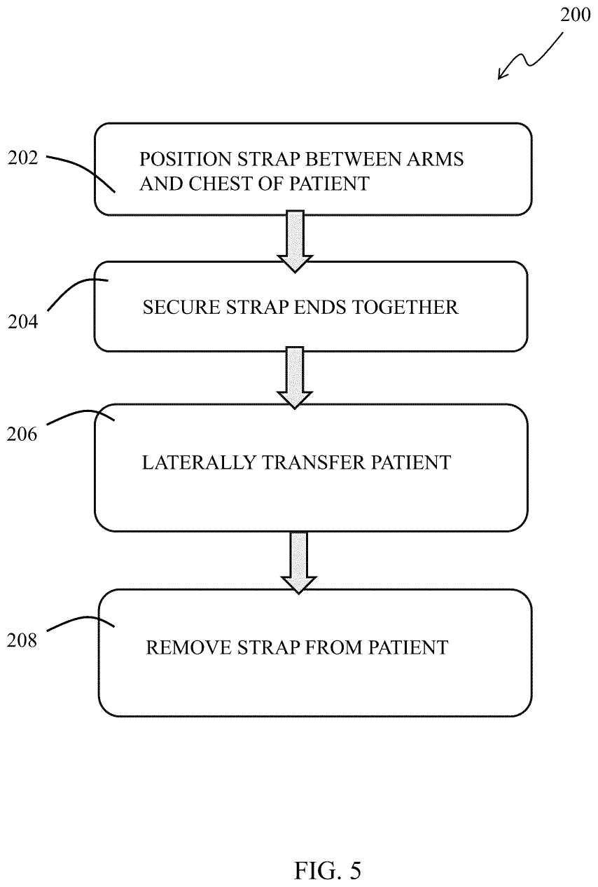

FIG. 5 shows a non-limiting, illustrative example of a method 200 of using the system 100 to assist with a lateral transfer of a patient 2, according to some aspects of the present disclosure. The method 200 can include a strap-positioning step 202 in which the strap 110 is positioned between the arms and chest of the patient with the arms resting on the patient's chest. The method 200 can further include a securement step 204 in which the strap 110 is bent into a hoop-like structure around the patient's arms and secured into the hoop-like structure using the adhesive system of the bilateral arm stabilizer system 100, as described herein. The method 200 can further include a transfer step 206 in which the lateral transfer of the patient is performed with the system 100 securing the arms of the patient on top of the chest of the patient. The method 200 can further include a strap-removal step 208 in which the frangible portion of the strap 110 is ruptured by applying a radial force to the hoop-like structure, as described herein. Once the system 100 has been ruptured out of the hoop-like structure, the strap 110 can be pulled out from under the arms of the patient to free the patient from the system 100.

### Other Variations and Terminology

While certain embodiments have been described, these embodiments have been presented by way of example only and are not intended to limit the scope of protection. Indeed, the novel methods and systems described herein may be embodied in a variety of other forms. It will be further understood by those skilled in the art that the present disclosure extends beyond the specifically disclosed embodiments to other alternative embodiments or uses and obvious modifications and equivalents thereof, including embodiments which do not provide all of the features and advantages set forth herein. Furthermore, various omissions, substitutions, and changes in the form of the methods and systems described herein may be made. Those skilled in the art will appreciate that in some embodiments, the actual steps taken in the processes illustrated or disclosed may differ from those shown in the figures. Depending on the embodiment, certain of the steps described above may be removed; others may be added. Accordingly, the scope of the present disclosure is not intended to be limited by the specific disclosures of preferred embodiments herein and may be defined by claims as presented herein or as presented in the future. The language of the claims is to be interpreted broadly based on the language employed in the claims and not limited to the examples described in the patent specification of during prosecution of the application, which examples are to be construed as non-exclusive.

Features, materials, characteristics, or groups described in conjunction with a particular aspect, embodiment, or example are to be understood to be applicable to any other aspect, embodiment, or example described herein unless incompatible therewith. All of the features disclosed in this specification (including any accompanying claims, abstract, and drawings), or all of the steps of any method or process so disclosed, may be combined in any combination, except combinations where at least some of such features or steps are mutually exclusive. The protection extends to any novel one, or any novel combination, of the features disclosed in this specification (including any accompanying claims, abstract, and drawings), or to any novel one, or any novel combination, of the steps of any method or process so disclosed.

Conditional language, such as “can,” “could,” “might,” or “may,” unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language, such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately,” “about,” “generally,” and “substantially” may refer to an amount that is within less than 10% of the stated amount. As another example, the terms “generally parallel” and “substantially parallel” may refer to a value, amount, or characteristic that departs from exactly parallel by less than 15 degrees.

What is claimed is:

1. A bilateral arm stabilizing system comprising:
a strap having a head end, a foot end, a front face, and a back face;
an anchoring pad disposed at the foot end;
an anchoring patch disposed at the head end, the anchoring patch configured to adhere to the anchoring pad to prevent the anchoring patch from moving relative to the anchoring pad; and
a bridge portion longitudinally aligned with a notch disposed on a lateral edge of the strap, the bridge portion having a transverse width dimension that is less than a transverse width of a distant portion of the strap that does not overlap longitudinally with the notch.

2. The strap of claim 1, wherein the bridge portion comprises a perforation that spans the bridge portion.

3. The strap of claim 1, wherein the distant portion comprises a perforation.

4. The strap of claim 1, wherein the bridge portion comprises a re-enforced portion, the re-enforced portion having a tensile strength greater than a tensile strength of the bridge portion.

5. The strap of claim 1, wherein the anchoring patch comprises one of a hook portion or a loop portion of a hook-and-loop material, and wherein the anchoring pad comprises the other of the hook portion or the loop portion.

6. The strap of claim 1, wherein the notch is sized to accommodate a finger being inserted into the notch.

7. The strap of claim 1, wherein the anchoring patch comprises an adhesive.

8. The strap of claim 1, wherein the anchoring pad comprises the front face of the strap.

9. The strap of claim 1, further comprising a perforation disposed between the notch and the head end.

10. The strap of claim 1, further comprising a perforation disposed between the notch and the foot end.

11. The strap of claim 10, wherein the perforation extends from a lateral edge of the strap without crossing a midline of the strap.

12. A method of attaching a bilateral arm stabilizing system to a patient whose arms are positioned on top of a chest of the patient, the method comprising:
positioning a strap having a head end and a foot end between the arms and chest such that the head end is disposed longitudinally between the arms and a head of the patient and the foot end is disposed longitudinally between the arms and a foot of the patient;
joining the head end with the foot end to form a hoop disposed circumferentially around the arms; and
securing the head end to the foot end to maintain the hoop, thereby attaching the bilateral arm stabilizing system to the patient.

13. The method of claim 12, wherein the head end and the foot end are secured together by an adhesive.

14. The method of claim 12, wherein the head end and the foot end are secured together by a hook-and-loop material.

15. The method of claim 12, further comprising aligning under the patient arms a notch cut into the strap.

16. The method of claim 12, further comprising aligning longitudinally between the head end and the arms a notch cut into the strap.

17. A method of performing a lateral bed transfer of a medical patient, the method comprising:
positioning a strap under the patient's arms resting on top of the patient's chest such that a head end of the strap is disposed between the patient's head and the patient's arms and a foot end of the strap is disposed between the patient's feet and the patient's arms;
forming a hoop-like structure with the strap by joining the head end to the foot end;
laterally moving the patient to an adjacent bed; and
releasing the strap by rupturing a frangible region of the strap.

18. The method of claim 17, wherein rupturing a frangible portion of the strap comprises breaking a perforation.
